(19) United States
(12) Patent Application Publication
(10) Pub. No.: US 2019/0144909 A1
(43) Pub. Date: May 16, 2019

(54) METHODS OF MODULATING BCKDH

(71) Applicant: NESTEC S.A., Vevey (CH)

(72) Inventors: Jerome Carayol, Pully (CH); Jorg Hager, Houtaud (FR); Armand Valesia, Chavannes-pres-Renens (CH)

(21) Appl. No.: 16/091,306

(22) PCT Filed: Apr. 6, 2017
(86) PCT No.: PCT/EP2017/058245

§ 371 Date: Oct. 4, 2018

(30) Foreign Application Priority Data
Apr. 6, 2016 (EP) 16164059.4

(51) Int. Cl. C12Q 1/00; G01N 33/50; C12N 15/52; C12N 9/02

(52) U.S. Cl. CPC C12Q 1/008; G01N 33/5008; C12N 15/52; C12Y 102/04004; G01N 2500/02; G01N 2500/20; C12N 9/0008

(57) ABSTRACT

An agent capable of increasing the activity of branched-chain alpha-keto acid dehydrogenase (BCKDH) for use in increasing leptin levels. The invention also relates to the use of such agents for supporting satiety and for supporting weight maintenance and/or treating or preventing obesity.

### FIELD OF THE INVENTION

The present invention relates to agents which are capable of modulating the activity of branched-chain alpha-keto acid dehydrogenase (BCKDH) and the use of such agents in therapy, in particular for use in increasing leptin levels. The invention also relates to the use of such agents for supporting satiety and for supporting weight maintenance and treating obesity. The invention also relates to methods of identifying such agents.

### BACKGROUND TO THE INVENTION

Obesity is a chronic metabolic disorder that has reached epidemic proportions in many areas of the world. Obesity is the major risk factor for serious co-morbidities such as type 2 diabetes mellitus, cardiovascular disease, dyslipidaemia and certain types of cancer (World Health Organ. Tech. Rep. Ser. (2000) 894: i-xii, 1-253).

Obesity refers to a condition in which an individual weighs more than usual as a result of excessive accumulation of energy from carbohydrate, fat and the like. The additional weight is typically retained in the form of fat under the skin or around the viscera.

Empirical data suggests that a weight loss of at least 10% of the initial weight results in a considerable decrease in the risk of obesity related co-morbidities (World Health Organ. Tech. Rep. Ser. (2000) 894: i-xii, 1-253). However, the capacity to lose weight shows large inter-subject variability.

Obesity is induced when the amount of energy intake exceeds the amount of energy consumed, thus in order to ameliorate obesity, a method of decreasing the amount of energy intake from fat, carbohydrate and the like or a method of increasing the amount of energy consumption by promoting in vivo metabolism is desired. Therefore, improvements in dietary habit and exercise are considered to be effective methods for the prevention and amelioration of obesity and obesity-related disorders. For example, it has long been recognised that low-calorie diet (LCD) interventions can be very efficient in reducing weight and that this weight loss is generally accompanied by an improvement in the risk of obesity related co-morbidities, in particular type 2 diabetes mellitus (World Health Organ. Tech. Rep. Ser. (2000) 894: i-xii, 1-253).

Although a number of methods are known for promoting weight loss, subjects face the risk of regaining lost weight once a period of weight loss intervention has been completed. Such regression risks reducing or potentially completely reversing any benefits that were associated with the loss of weight.

Accordingly, there remains a significant need not only for improved methods of promoting weight loss, but also for methods for supporting weight maintenance (preventing or reducing the regain of lost weight, and hence supporting maintenance of weight at a level similar to that achieved following weight loss intervention). Such improvements would provide more complete treatments for obesity, thus decreasing the risk of obesity-related disorders. In particular, there is a need for methods that can be successfully applied in the period following weight loss so as to reduce the risk of regain of any weight lost.

Leptin is generally understood to be linked to appetite suppression and control. The inventors of the present application have surprisingly observed a link between leptin expression levels and the regulatory region of the BCKDHB gene.

### SUMMARY OF THE INVENTION

The inventors carried out an analysis of protein quantitative trait loci (pQTL) on weight loss intervention data obtained from the Diogenes study. This study is a pan-European, randomised and controlled dietary intervention study investigating the effects of dietary protein and glycaemic index on weight loss and weight maintenance in obese and overweight families in eight European centres (Larsen et al. (2009) Obesity Rev. 11: 76-91). In brief, the Diogenes study subjected screened participants to a low-calorie diet (LCD) phase (CID1), in which overweight/obese subjects followed an 8 week Modifast® diet (approximately 800 kCal/day), followed by a weight maintenance phase (CID2).

In the present context, the pQTL are genomic loci that contribute to variations in protein levels during the LCD phase. The inventors specifically analysed pQTL associated with proteins which exhibited expression changes that correlated with weight loss.

The inventors observed differential expression of leptin during the LCD phase that was significantly associated with weight loss, a finding which correlates with the understanding in the field that leptin is linked to appetite suppression and control. The inventors also observed that the best pQTL associated with leptin differential expression is located in the regulatory region of the BCKDHB gene.

BCKDHB encodes the branched-chain alpha-keto acid dehydrogenase E1 B subunit, which is part of an enzyme complex involved in the breakdown of the branched-chain amino acids (BCAAs) leucine, isoleucine and valine. BCAAs themselves have been linked with appetite suppression, thus two independent links have been established between BCKDHB and appetite suppression and control.

Accordingly, the inventors have established a significant relationship between branched-chain alpha-keto acid dehydrogenase (BCKDH) and appetite suppression and control, thus providing for new interventions to support weight maintenance and the treatment of obesity.

Accordingly, in one aspect the invention provides an agent capable of increasing the activity of branched-chain alpha-keto acid dehydrogenase (BCKDH) for use in increasing levels of leptin.

In another aspect, the invention provides an agent capable of increasing the activity of branched-chain alpha-keto acid dehydrogenase (BCKDH) for use in suppressing the appetite of a subject. In another aspect, the invention provides an agent capable of increasing the activity of branched-chain alpha-keto acid dehydrogenase (BCKDH) for use in supporting or prolonging satiety. In another aspect, the invention provides an agent capable of increasing the activity of branched-chain alpha-keto acid dehydrogenase (BCKDH) for use in reducing food intake by a subject. In another aspect, the invention provides an agent capable of increasing the activity of branched-chain alpha-keto acid dehydrogenase (BCKDH) for use in reducing fat deposition in a subject.

In another aspect, the invention provides the use of an agent capable of increasing the activity of branched-chain alpha-keto acid dehydrogenase (BCKDH) for supporting weight maintenance. In another aspect, the invention provides the use of an agent capable of increasing the activity of branched-chain alpha-keto acid dehydrogenase (BCKDH) for treating or preventing obesity. In another aspect, the invention provides the use of an agent capable of increasing the activity of branched-chain alpha-keto acid dehydrogenase (BCKDH) for suppressing the appetite of a subject. In another aspect, the invention provides the use of an agent capable of increasing the activity of branched-chain alpha-keto acid dehydrogenase (BCKDH) for prolonging satiety. In another aspect, the invention provides the use of an agent capable of increasing the activity of branched-chain alpha-keto acid dehydrogenase (BCKDH) for reducing food intake by a subject. In another aspect, the invention provides the use of an agent capable of increasing the activity of branched-chain alpha-keto acid dehydrogenase (BCKDH) for reducing fat deposition in a subject.

In another aspect, the invention provides a method of supporting weight maintenance comprising administering an agent of the invention to a subject in need thereof. In another aspect, the invention provides a method of suppressing the appetite of a subject comprising administering an agent of the invention to a subject in need thereof. In another aspect, the invention provides a method of prolonging satiety comprising administering an agent of the invention to a subject in need thereof. In another aspect, the invention provides a method of reducing food intake by a subject comprising administering an agent of the invention to a subject in need thereof. In another aspect, the invention provides a method of reducing fat deposition in a subject comprising administering an agent of the invention to a subject in need thereof. In another aspect, the invention provides a method of treating or preventing obesity comprising administering an agent of the invention to a subject in need thereof.

In one embodiment, the agent increases the activity of the BCKDH E1 B subunit.

The activity of BCKDH and/or the BCKDH E1 B subunit may be increased in comparison with the activity in the absence of the agent of the invention. The activity of BCKDH (in particular the BCKDH E1 B subunit) may be increased by, for example, at least 1%, 2%, 3%, 4%, 5%, 6%, 7%, 8%, 9%, 10%, 15%, 20%, 25%, 50%, 75%, 100% or more.

The BCKDH activity (in particular the BCKDH E1 B subunit) may be increased, for example, through the use of agents which exhibit an agonistic effect on BCKDH (in particular the BCKDH E1 B subunit) or which increase the level of BCKDH (in particular the BCKDH E1 B subunit) in a cell.

In one embodiment, the agent increases or prolongs satiety. In another embodiment, the agent reduces food intake by a subject. In another embodiment, the agent reduces fat deposition in a subject.

In one embodiment, the agent is administered to a subject during or after a weight loss intervention, preferably after a weight loss intervention. The weight loss intervention may be, for example, a diet regimen (e.g. a low-calorie diet) and/or an exercise regimen.

In one embodiment, the agent increases the level of BCKDH in a subject. Preferably, the agent increases the level of BCKDH E1 B subunit in a subject. In this context, “level” refers to the amount of BCKDH or the BCKDH E1 B subunit and may be measured, for example, by analysing the amount of protein expressed and/or by analysing the amount of the corresponding mRNA present. Preferably, the agent increases the expression of BCKDH and/or the BCKDH E1 B subunit.

For example, polynucleotides encoding BCKDH (in particular the BCKDH E1 B and/or A subunits) may be introduced into a cell to provide for expression of the encoded polypeptides by the cell. Thus, the agent of the invention may be in the form of a polynucleotide encoding BCKDH (in particular the BCKDH E1 B and/or A subunits). Preferably, the polynucleotide is in the form of a vector, such as a viral vector.

The level of BCKDH and/or BCKDH E1 B subunit may be increased in comparison with the level in the absence of the agent of the invention. The level of BCKDH (in particular the BCKDH E1 B subunit) may be increased by, for example, at least 1%, 2%, 3%, 4%, 5%, 6%, 7%, 8%, 9%, 10%, 15%, 20%, 25%, 50%, 75%, 100% or more.

In one embodiment, the agent does not affect the activity of branched-chain alpha-keto acid dehydrogenase kinase (BCKDH kinase). In one embodiment, the agent is not fenofibrate.

In one embodiment, the agent is selected from resveratrol or valproic acid, or plant bioactives. In one embodiment, the agent is selected from the agents listed in Table 1a. In one embodiment, the agent is 2,4-dinitrotoluene. In one embodiment, the agent is Ammonium Chloride. In one embodiment, the agent is Benzo(a)pyrene. In one embodiment, the agent is Cuprizone. In one embodiment, the agent is Diethylnitrosamine. In one embodiment, the agent is Methylmercuric chloride. In one embodiment, the agent is pirinixic acid. In one embodiment, the agent is potassium chromate(VI). In one embodiment, the agent is Tetrachlorodibenzodioxin. In one embodiment, the agent is selected from the agents listed in Table 1b. In one embodiment, the agent is 1,12-benzoperylene. In one embodiment, the agent is 17-ethynyl-5-androstene-3, 7, 17-triol. In one embodiment, the agent is 2,4-dinitrotoluene. In one embodiment, the agent is Acetaminophen. In one embodiment, the agent is Amiodarone. In one embodiment, the agent is Ammonium Chloride. In one embodiment, the agent is Atrazine. In one embodiment, the agent is Bisphenol A. In one embodiment, the agent is Carbamazepine. In one embodiment, the agent is Carbon Tetrachloride. In one embodiment, the agent is Chloroprene. In one embodiment, the agent is Clofibrate. In one embodiment, the agent is Ethinyl Estradiol. In one embodiment, the agent is Fluorouracil. In one embodiment, the agent is Furan. In one embodiment, the agent is Ketamine. In one embodiment, the agent is Pirinixic acid. In one embodiment, the agent is Streptozocin. In one embodiment, the agent is Tetracycline. In one embodiment, the agent is Topotecan. In one embodiment, the agent is Tunicamycin. In one embodiment, the agent is Vancomycin. In one embodiment, the agent is Vinclozolin.

In one embodiment, the agent decreases the activity of branched-chain alpha-keto acid dehydrogenase kinase (BCKDH kinase).

The activity of BCKDH kinase may be decreased in comparison with the activity in the absence of the agent of the invention. The activity of BCKDH kinase may be decreased by, for example, at least 1%, 2%, 3%, 4%, 5%, 6%, 7%, 8%, 9%, 10%, 15%, 20%, 25%, 50%, 75% or 100%.

The agent may be, for example, a BCKDH kinase antagonist or inhibitor. Preferably, the agent is selected from the group consisting of an siRNA, shRNA, miRNA, antisense RNA, polynucleotide, polypeptide or small molecule. The polypeptide may be, for example, an antibody. Thus, the agent of the invention may be in the form of a polynucleotide encoding an siRNA, shRNA, miRNA or antisense RNA that targets BCKDH kinase, or a polypeptide (e.g. an antibody). Preferably, the polynucleotide is in the form of a vector, such as a viral vector.

In one embodiment, the agent decreases the level of BCKDH kinase. In this context, “level” refers to the amount of BCKDH kinase and may be measured, for example, by analysing the amount of protein expressed and/or by analysing the amount of the corresponding mRNA present. Preferably, the agent decreases the expression of BCKDH kinase. For example, siRNAs, shRNAs, miRNAs or antisense RNAs may reduce expression of BCKDH kinase.

The level of BCKDH kinase may be decreased in comparison with the level in the absence of the agent of the invention. The level of BCKDH kinase may be decreased by, for example, at least 1%, 2%, 3%, 4%, 5%, 6%, 7%, 8%, 9%, 10%, 15%, 20%, 25%, 50%, 75% or 100%.

In one embodiment, the agent is α-chloroisocaproic acid or α-ketoisocaproic acid (KIC), or is selected from the agents listed in Table 2.

The agent of the invention may be an agent identified by a method of the invention.

In another aspect, the invention provides a method of identifying an agent capable of supporting weight maintenance and/or treating or preventing obesity in a subject comprising the steps:
(a) contacting a preparation comprising a branched-chain alpha-keto acid dehydrogenase (BCKDH) polypeptide or polynucleotide with a candidate agent; and
(b) detecting whether the candidate agent affects the activity of the BCKDH polypeptide or polynucleotide.

The effect on activity of the BCKDH polypeptide or polynucleotide may be analysed by comparing the activities of the BCKDH polypeptide or polynucleotide in the presence and absence (i.e. a control experiment) of the candidate agent.

In one embodiment, the BCKDH is the BCKDH E1 B subunit.

In one embodiment, the method comprises contacting the preparation comprising BCKDH with a candidate agent and measuring the conversion of NAD+ to NADH. The conversion of NAD⁺ to NADH may be analysed spectrophotometrically.

In another aspect, the invention provides a method of identifying an agent capable of supporting weight maintenance and/or treating or preventing obesity in a subject comprising the steps:
(a) contacting a preparation comprising a branched-chain alpha-keto acid dehydrogenase kinase (BCKDH kinase) polypeptide or polynucleotide with a candidate agent; and
(b) detecting whether the candidate agent affects the activity of the BCKDH kinase polypeptide or polynucleotide.

The effect on activity of the BCKDH kinase polypeptide or polynucleotide may be analysed by comparing the activities of the BCKDH kinase polypeptide or polynucleotide in the presence and absence (i.e. a control experiment) of the candidate agent.

In one embodiment, the method comprises contacting the preparation comprising BCKDH kinase with a candidate agent in the presence of ATP and measuring the incorporation of phosphate into a substrate or measuring the conversion of ATP to ADP.

In another aspect, the invention provides a method of identifying an agent that increases the activity of branched-chain alpha-keto acid dehydrogenase (BCKDH) comprising the steps:
(a) contacting a preparation comprising a BCKDH polypeptide or polynucleotide with a candidate agent; and
(b) detecting whether the candidate agent affects the activity of the BCKDH polypeptide or polynucleotide.

In one embodiment, the BCKDH is the BCKDH E1 B subunit.

In one embodiment, the method comprises contacting the preparation comprising BCKDH with a candidate agent and measuring the conversion of NAD⁺ to NADH. The conversion of NAD⁺ to NADH may be analysed spectrophotometrically.

In another aspect, the invention provides a method of identifying an agent that decreases the activity of branched-chain alpha-keto acid dehydrogenase kinase (BCKDH kinase) comprising the steps:
(a) contacting a preparation comprising a BCKDH kinase polypeptide or polynucleotide with a candidate agent; and
(b) detecting whether the candidate agent affects the activity of the BCKDH kinase polypeptide or polynucleotide.

In one embodiment, the method comprises contacting the preparation comprising BCKDH kinase with a candidate agent in the presence of ATP and measuring the incorporation of phosphate into a substrate or measuring the conversion of ATP to ADP.

In another aspect, the invention provides a method of identifying an agent that increases the expression or processing of branched-chain alpha-keto acid dehydrogenase (BCKDH), preferably the BCKDH E1 B subunit, comprising the steps:
(a) contacting a cell, preferably a cell expressing the BCKDH, with a candidate agent; and
(b) detecting whether the candidate agent increases the expression or processing of the BCKDH.

In another aspect, the invention provides a method of identifying an agent that decreases the expression or processing of branched-chain alpha-keto acid dehydrogenase kinase (BCKDH kinase) comprising the steps:
(a) contacting a cell expressing the BCKDH kinase with a candidate agent; and
(b) detecting whether the candidate agent decreases the expression or processing of the BCKDH kinase.

The methods of the invention may be methods for identifying an agent capable of suppressing the appetite of a subject, increasing or prolonging satiety, reducing food intake by a subject and/or reducing fat deposition in a subject.

In another aspect, the invention provides the use of branched-chain alpha-keto acid dehydrogenase (BCKDH), in particular the BCKDH E1 B subunit, or BCKDH kinase, or a polynucleotide encoding the same, in a method of identifying an agent that supports weight maintenance, suppresses the appetite of a subject, increases or prolongs satiety, reduces food intake by a subject, reduces fat deposition in a subject, and/or treats or prevents obesity.

In another aspect, the invention provides the use of an agent capable of increasing the activity of branched-chain alpha-keto acid dehydrogenase (BCKDH) for manufacturing a medicament for use in supporting weight maintenance, suppressing the appetite of a subject, increasing or prolonging satiety, reducing food intake by a subject, reducing fat deposition in a subject, and/or treating or preventing obesity.

### DESCRIPTION OF THE DRAWINGS

FIG. 1

A Manhattan plot zooming in on a region located in the regulatory region of the BCKDHB gene of chromosome 6.

FIG. 2

Box plots indicating that protein expression stratified based on trans-acting SNP genotype did not underline a strong difference of expression.

FIG. 3

Variables distribution for leucine, isoleucine and valine before (1) and after (2) low caloric diet (LCD) intervention.

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### Branched-Chain Alpha-Keto Acid Dehydrogenase (BCKDH)

Branched-chain alpha-keto acid dehydrogenase (BCKDH) is a protein complex located on the mitochondrial inner membrane which plays a key role in the catabolism of the branched-chain amino acids (BCAAs) valine, leucine and isoleucine. In BCAA catabolism, the BCKDH complex catalyses the oxidative decarboxylation of the branched-chain alpha-keto acid, which is a rate limiting step in the overall catabolic pathway.

The BCKDH complex consists of three subunits:
1. E1 subunit (EC 1.2.4.4), which exhibits branched-chain alpha-keto acid decarboxylase activity. The E1 subunit is comprised of A and B chains (also known as a and 3, respectively), which are encoded by the BCKDHA and BCKDHB genes;
2. E2 subunit (EC 2.3.1.168), which exhibits lipoamide acyltransferase activity; and
3. E3 subunit (EC 1.8.1.4), which exhibits lipoamide dehydrogenase activity.

The E2 subunit acts as the core of the BCKDH complex and is found in either 24 copies in octahedral symmetry or in 60 copies in icosahedral symmetry. Subunits E1 and E3 bind to E2 via non-covalent bonds, each with multiple copies.

Mutations in the BCKDH complex, in particular mutations located in the E1 B subunit have been associated with many disorders in humans, including Maple Syrup Urine Disease (MSUP; Ævarsson, A. et al. (2000) Structure 8: 277-291).

In one embodiment, the BCKDH is human BCKDH.

An example amino acid sequence of the BCKDH E1 B subunit is the sequence deposited under NCBI Accession No. NP 000047.1.

An example amino acid sequence of the BCKDH E1 B subunit is:

(SEQ ID NO: 1)
MAVVAAAAGWLLRLRAAGAEGHWRRLPGAGLARGFLHPAATVEDAAQ
RRQVAHFTFQPDPEPREYGQTQKMNLFQSVTSALDNSLAKDPTAVIF
GEDVAFGGVFRCTVGLRDKYGKDRVFNTPLCEQGIVGFGIGIAVTGA
TAIAEIQFADYIFPAFDQIVNEAAKYRYRSGDLFNCGSLTIRSPWGC
VGHGALYHSQSPEAFFAHCPGIKVVIPRSPFQAKGLLLSCIEDKNPC
IFFEPKILYRAAAEEVPIEPYNIPLSQAEVIQEGSDVTLVAWGTQVH
VIREVASMAKEKLGVSCEVIDLRTIIPWDVDTICKSVIKTGRLLISH
EAPLTGGFASEISSTVQEECFLNLEAPISRVCGYDTPFPHIFEPFYI
PDKWKCYDALRKMINY

An example nucleotide sequence encoding the BCKDH E1 B subunit is the sequence deposited under NCBI Accession No. NM_000056.4.

An example nucleotide sequence encoding the BCKDH E1 B subunit is:

(SEQ ID NO: 2)
ATGGCGGTTGTAGCGGCGGCTGCCGGCTGGCTACTCAGGCTCAGGGC
GGCAGGGGCTGAGGGGCACTGGCGTCGGCTTCCTGGCGCGGGGCTGG
CGCGGGGCTTTTTGCACCCCGCCGCGACTGTCGAGGATGCGGCCCAG
AGGCGGCAGGTGGCTCATTTTACTTTCCAGCCAGATCCGGAGCCCCG
GGAGTACGGGCAAACTCAGAAAATGAATCTTTTCCAGTCTGTAACAA
GTGCCTTGGATAACTCATTGGCCAAAGATCCTACTGCAGTAATATTT
GGTGAAGATGTTGCCTTTGGTGGAGTCTTTAGATGCACTGTTGGCTT
GCGAGACAAATATGGAAAAGATAGAGTTTTTAATACCCCATTGTGTG
AACAAGGAATTGTTGGATTTGGAATCGGAATTGCGGTCACTGGAGCT
ACTGCCATTGCGGAAATTCAGTTTGCAGATTATATTTTCCCTGCATT
TGATCAGATTGTTAATGAAGCTGCCAAGTATCGCTATCGCTCTGGGG
ATCTTTTTAACTGTGGAAGCCTCACTATCCGGTCCCCTTGGGGCTGT
GTTGGTCATGGGGCTCTCTATCATTCTCAGAGTCCTGAAGCATTTTT
TGCCCATTGCCCAGGAATCAAGGTGGTTATACCCAGAAGCCCTTTCC
AGGCCAAAGGACTTCTTTTGTCATGCATAGAGGATAAAAATCCTTGT
ATATTTTTTGAACCTAAAATACTTTACAGGGCAGCAGCGGAAGAAGT
CCCTATAGAACCATACAACATCCCACTGTCCCAGGCCGAAGTCATAC
AGGAAGGGAGTGATGTTACTCTAGTTGCCTGGGGCACTCAGGTTCAT
GTGATCCGAGAGGTAGCTTCCATGGCAAAAGAAAAGCTTGGAGTGTC
TTGTGAAGTCATTGATCTGAGGACTATAATACCTTGGGATGTGGACA
CAATTTGTAAGTCTGTGATCAAAACAGGGCGACTGCTAATCAGTCAC
GAGGCTCCCTTGACAGGCGGCTTTGCATCGGAAATCAGCTCTACAGT
TCAGGAGGAATGTTTCTTGAACCTAGAGGCTCCTATATCAAGAGTAT
GTGGTTATGACACACCATTTCCTCACATTTTTGAACCATTCTACATC
CCAGACAAATGGAAGTGTTATGATGCCCTTCGAAAAATGATCAACTA
TTGA

In one embodiment, the BCKDH E1 B subunit comprises an amino acid sequence that has at least 70%, 80%, 85%, 90%, 95%, 96%, 97%, 98%, 99% or 100% identity to SEQ ID NO: 1, preferably wherein the amino acid sequence substantially retains the natural function of the protein represented by SEQ ID NO: 1.

In one embodiment, the BCKDH E1 B subunit-encoding nucleotide sequence comprises a nucleotide sequence that has at least 70%, 80%, 85%, 90%, 95%, 96%, 97%, 98%, 99% or 100% identity to SEQ ID NO: 2, preferably wherein the protein encoded by the nucleotide sequence substantially retains the natural function of the protein represented by SEQ ID NO: 1.

In one embodiment, the BCKDH E1 B subunit-encoding nucleotide sequence comprises a nucleotide sequence that encodes an amino acid sequence that has at least 70%, 80%, 85%, 90%, 95%, 96%, 97%, 98%, 99% or 100% identity to SEQ ID NO: 1, preferably wherein the amino acid sequence substantially retains the natural function of the protein represented by SEQ ID NO: 1.

An example amino acid sequence of the BCKDH E1 A subunit is the sequence deposited under NCBI Accession No. NP 000700.1.

An example amino acid sequence of the BCKDH E1 A subunit is:

(SEQ ID NO: 3)
MAVAIAAARVWRLNRGLSQAALLLLRQPGARGLARSHPPRQQQQFSS
LDDKPQFPGASAEFIDKLEFIQPNVISGIPIYRVMDRQGQIINPSED
PHLPKEKVLKLYKSMTLLNTMDRILYESQRQGRISFYMTNYGEEGTH
VGSAAALDNTDLVFGQYREAGVLMYRDYPLELFMAQCYGNISDLGKG
RQMPVHYGCKERHFVTISSPLATQIPQAVGAAYAAKRANANRVVICY
FGEGAASEGDAHAGFNFAATLECPIIFFCRNNGYAISTPTSEQYRGD
GIAARGPGYGIMSIRVDGNDVFAVYNATKEARRRAVAENQPFLIEAM
TYRIGHHSTSDDSSAYRSVDEVNYWDKQDHPISRLRHYLLSQGWWDE
EQEKAWRKQSRRKVMEAFEQAERKPKPNPNLLFSDVYQEMPAQLRKQ
QESLARHLQTYGEHYPLDHFDK

An example nucleotide sequence encoding the BCKDH E1 A subunit is the sequence deposited under NCBI Accession No. NM_000709.3.

An example nucleotide sequence encoding the BCKDH E1 A subunit is:

(SEQ ID NO: 4)
ATGGCGGTAGCGATCGCTGCAGCGAGGGTCTGGCGGCTAAACCGTGG
TTTGAGCCAGGCTGCCCTCCTGCTGCTGCGGCAGCCTGGGGCTCGGG
GACTGGCTAGATCTCACCCCCCCAGGCAGCAGCAGCAGTTTTCATCT
CTGGATGACAAGCCCCAGTTCCCAGGGGCCTCGGCGGAGTTTATAGA
TAAGTTGGAATTCATCCAGCCCAACGTCATCTCTGGAATCCCCATCT
ACCGCGTCATGGACCGGCAAGGCCAGATCATCAACCCCAGCGAGGAC
CCCCACCTGCCGAAGGAGAAGGTGCTGAAGCTCTACAAGAGCATGAC
ACTGCTTAACACCATGGACCGCATCCTCTATGAGTCTCAGCGGCAGG
GCCGGATCTCCTTCTACATGACCAACTATGGTGAGGAGGGCACGCAC
GTGGGGAGTGCCGCCGCCCTGGACAACACGGACCTGGTGTTTGGCCA
GTACCGGGAGGCAGGTGTGCTGATGTATCGGGACTACCCCCTGGAAC
TATTCATGGCCCAGTGCTATGGCAACATCAGTGACTTGGGCAAGGGG
CGCCAGATGCCTGTCCACTACGGCTGCAAGGAACGCCACTTCGTCAC
TATCTCCTCTCCACTGGCCACGCAGATCCCTCAGGCGGTGGGGGCGG
CGTACGCAGCCAAGCGGGCCAATGCCAACAGGGTCGTCATCTGTTAC
TTCGGCGAGGGGGCAGCCAGTGAGGGGGACGCCCATGCCGGCTTCAA
CTTCGCTGCCACACTTGAGTGCCCCATCATCTTCTTCTGCCGGAACA
ATGGCTACGCCATCTCCACGCCCACCTCTGAGCAGTATCGCGGCGAT
GGCATTGCAGCACGAGGCCCCGGGTATGGCATCATGTCAATCCGCGT
GGATGGTAATGATGTGTTTGCCGTATACAACGCCACAAAGGAGGCCC
GACGGCGGGCTGTGGCAGAGAACCAGCCCTTCCTCATCGAGGCCATG
ACCTACAGGATCGGGCACCACAGCACCAGTGACGACAGTTCAGCGTA
CCGCTCGGTGGATGAGGTCAATTACTGGGATAAACAGGACCACCCCA
TCTCCCGGCTGCGGCACTATCTGCTGAGCCAAGGCTGGTGGGATGAG
GAGCAGGAGAAGGCCTGGAGGAAGCAGTCCCGCAGGAAGGTGATGGA
GGCCTTTGAGCAGGCCGAGCGGAAGCCCAAACCCAACCCCAACCTAC
TCTTCTCAGACGTGTATCAGGAGATGCCCGCCCAGCTCCGCAAGCAG
CAGGAGTCTCTGGCCCGCCACCTGCAGACCTACGGGGAGCACTACCC
ACTGGATCACTTCGATAAGTGA

In one embodiment, the BCKDH E1 A subunit comprises an amino acid sequence that has at least 70%, 80%, 85%, 90%, 95%, 96%, 97%, 98%, 99% or 100% identity to SEQ ID NO: 3, preferably wherein the amino acid sequence substantially retains the natural function of the protein represented by SEQ ID NO: 3.

In one embodiment, the BCKDH E1 A subunit-encoding nucleotide sequence comprises a nucleotide sequence that has at least 70%, 80%, 85%, 90%, 95%, 96%, 97%, 98%, 99% or 100% identity to SEQ ID NO: 4, preferably wherein the protein encoded by the nucleotide sequence substantially retains the natural function of the protein represented by SEQ ID NO: 3.

In one embodiment, the BCKDH E1 A subunit-encoding nucleotide sequence comprises a nucleotide sequence that encodes an amino acid sequence that has at least 70%, 80%, 85%, 90%, 95%, 96%, 97%, 98%, 99% or 100% identity to SEQ ID NO: 3, preferably wherein the amino acid sequence substantially retains the natural function of the protein represented by SEQ ID NO: 3.

The BCKDH E1 A and B subunits are mitochondrial proteins and their sequences may include mitochondrial-targeting signal sequences. Such signal sequences may be cleaved upon targeting of the protein to the mitochondrion, thus the protein may naturally exist in a mature form lacking the signal sequence. The skilled person is readily able to determine such signal sequences using appropriate bioinformatic and molecular biology techniques. For example, residues 1-50 of SEQ ID NO: 1 and residues 1-45 of SEQ ID NO: 3 may act as signal sequences.

### Branched-Chain Alpha-Keto Acid Dehydrogenase Kinase (BCKDH Kinase)

The activity of BCKDH is regulated by a phosphorylation/dephosphorylation cycle. Branched-chain alpha-keto acid dehydrogenase kinase (BCKDH kinase) inactivates the BCKDH complex by phosphorylation of the BCKDH E1 A subunit, while BCKDH phosphatase activates the complex by dephosphorylating the BCKDH E1 A subunit.

In one embodiment, the BCKDH kinase is human BCKDH kinase.

An example amino acid sequence of the BCKDH kinase is the sequence deposited under NCBI Accession No. NP_005872.2.

An example amino acid sequence of the BCKDH kinase is:

(SEQ ID NO: 5)
MILASVLRSGPGGGLPLRPLLGPALALRARSTSATDTHHVEMARERS
KTVTSFYNQSAIDAAAEKPSVRLTPTMMLYAGRSQDGSHLLKSARYL
QQELPVRIAHRIKGFRCLPFIIGCNPTILHVHELYIRAFQKLTDFPP
IKDQADEAQYCQLVRQLLDDHKDVVTLLAEGLRESRKHIEDEKLVRY
FLDKTLTSRLGIRMLATHHLALHEDKPDFVGIICTRLSPKKIIEKWV
DFARRLCEHKYGNAPRVRINGHVAARFPFIPMPLDYILPELLKNAMR
ATMESHLDTPYNVPDVVITIANNDVDLIIRISDRGGGIAHKDLDRVM
DYHFTTAEASTQDPRISPLFGHLDMHSGAQSGPMHGFGFGLPTSRAY
AEYLGGSLQLQSLQGIGTDVYLRLRHIDGREESFRI

An example nucleotide sequence encoding the BCKDH kinase is the sequence deposited under NCBI Accession No. NM 005881.3.

An example nucleotide sequence encoding the BCKDH kinase is:

(SEQ ID NO: 6)
ATGATCCTGGCGTCGGTGCTGAGGAGCGGTCCCGGGGGCGGGCTTCC
GCTCCGGCCCCTCCTGGGACCCGCACTCGCGCTCCGGGCCCGCTCGA
CGTCGGCCACCGACACACACCACGTGGAGATGGCTCGGGAGCGCTCC
AAGACCGTCACCTCCTTTTACAACCAGTCGGCCATCGACGCGGCAGC
GGAGAAGCCCTCAGTCCGCCTAACGCCCACCATGATGCTCTACGCTG
GCCGCTCTCAGGACGGCAGCCACCTTCTGAAAAGTGCTCGGTACCTG
CAGCAAGAACTTCCAGTGAGGATTGCTCACCGCATCAAGGGCTTCCG
CTGCCTTCCTTTCATCATTGGCTGCAACCCCACCATACTGCACGTGC
ATGAGCTATATATCCGTGCCTTCCAGAAGCTGACAGACTTCCCTCCG
ATCAAGGACCAGGCGGACGAGGCCCAGTACTGCCAGCTGGTGCGACA
GCTGCTGGATGACCACAAGGATGTGGTGACCCTCTTGGCAGAGGGCC
TACGTGAGAGCCGGAAGCACATAGAGGATGAAAAGCTCGTCCGCTAC
TTCTTGGACAAGACGCTGACTTCGAGGCTTGGAATCCGCATGTTGGC
CACGCATCACCTGGCGCTGCATGAGGACAAGCCTGACTTTGTCGGCA
TCATCTGTACTCGTCTCTCACCAAAGAAGATTATTGAGAAGTGGGTG
GACTTTGCCAGACGCCTGTGTGAGCACAAGTATGGCAATGCGCCCCG
TGTCCGCATCAATGGCCATGTGGCTGCCCGGTTCCCCTTCATCCCTA
TGCCACTGGACTACATCCTGCCGGAGCTGCTCAAGAATGCCATGAGA
GCCACAATGGAGAGTCACCTAGACACTCCCTACAATGTCCCAGATGT
GGTCATCACCATCGCCAACAATGATGTCGATCTGATCATCAGGATCT
CAGACCGTGGTGGAGGAATCGCTCACAAAGATCTGGACCGGGTCATG
GACTACCACTTCACTACTGCTGAGGCCAGCACACAGGACCCCCGGAT
CAGCCCCCTCTTTGGCCATCTGGACATGCATAGTGGCGCCCAGTCAG
GACCCATGCACGGCTTTGGCTTCGGGTTGCCCACGTCACGGGCCTAC
GCGGAGTACCTCGGTGGGTCTCTGCAGCTGCAGTCCCTGCAGGGCAT
TGGCACGGACGTCTACCTGCGGCTCCGCCACATCGATGGCCGGGAGG
AAAGCTTCCGGATCTGA

In one embodiment, the BCKDH kinase comprises an amino acid sequence that has at least 70%, 80%, 85%, 90%, 95%, 96%, 97%, 98%, 99% or 100% identity to SEQ ID NO: 5, preferably wherein the amino acid sequence substantially retains the natural function of the protein represented by SEQ ID NO: 5.

In one embodiment, the BCKDH kinase-encoding nucleotide sequence comprises a nucleotide sequence that has at least 70%, 80%, 85%, 90%, 95%, 96%, 97%, 98%, 99% or 100% identity to SEQ ID NO: 6, preferably wherein the protein encoded by the nucleotide sequence substantially retains the natural function of the protein represented by SEQ ID NO: 5.

In one embodiment, the BCKDH kinase-encoding nucleotide sequence comprises a nucleotide sequence that encodes an amino acid sequence that has at least 70%, 80%, 85%, 90%, 95%, 96%, 97%, 98%, 99% or 100% identity to SEQ ID NO: 5, preferably wherein the amino acid sequence substantially retains the natural function of the protein represented by SEQ ID NO: 5.

BCKDH kinase is a mitochondrial protein and its sequence may include a mitochondrial-targeting signal sequence. Such a signal sequence may be cleaved upon targeting of the protein to the mitochondrion, thus the protein may naturally exist in a mature form lacking the signal sequence. The skilled person is readily able to determine such signal sequences using appropriate bioinformatic and molecular biology techniques. For example, residues 1-30 of SEQ ID NO: 5 may act as a signal sequence.

### Weight Loss and Weight Maintenance

“Weight loss” may refer to a reduction in parameters such as weight (e.g. in kilograms), body mass index (kg/m²), waist-hip ratio (e.g. in centimetres), fat mass (e.g. in kilograms), hip circumference (e.g. in centimetres) or waist circumference (e.g. in centimetres).

Weight loss may be calculated by subtracting the value of one or more of the aforementioned parameters at the end of an intervention (e.g. a diet and/or exercise regimen) from the value of the parameter at the onset of the intervention.

The degree of weight loss may be expressed as a percent change of one of the aforementioned weight phenotype parameters (e.g. a percent change in a subject's body weight (e.g. in kilograms) or body mass index (kg/m²)). For example, a subject may lose at least 10% of their initial body weight, at least 8% of their initial body weight, or at least 5% of their initial body weight. By way of example only, a subject may lose between 5 and 10% of their initial body weight.

In one embodiment, a degree of weight loss of at least 10% of initial body weight results in a considerable decrease in the risk of obesity-related co-morbidities.

“Weight maintenance” may refer to the maintenance in parameters such as weight (e.g. in kilograms), body mass index (kg/m²), waist-hip ratio (e.g. in centimetres) fat mass (e.g. in kilograms), hip circumference (e.g. in centimetres) or waist circumference (e.g. in centimetres). Weight maintenance may refer to, for example, maintaining weight lost following an intervention (e.g. a diet and/or exercise regimen).

The degree of weight maintenance may be calculated by determining the change in one or more of the afore-mentioned parameters over a period of time. The period of time may be, for example, at least 5, 10, 15, 20, 25, 30, 35, 40, 45 or 50 weeks.

Weight maintenance supported by the agents of the invention may result in, for example, a change (e.g. gain) of less than 10%, 9%, 8%, 7%, 6%, 5%, 4%, 3%, 2% or 1% in one or more of the afore-mentioned parameters over a period of time.

The degree of weight maintenance may be expressed as the weight regained during a period following attainment of weight loss, for example as a percentage of the weight lost during attainment of weight loss.

Weight maintenance supported by the agents of the invention may result through suppression of a subject's appetite following administration of the agent. The subject may therefore have a reduced appetite compared to the appetite in the absence of the agent of the invention.

Weight maintenance supported by the agents of the invention may result through control of a subject's appetite following administration of the agent. The subject may therefore maintain control over their appetite and therefore maintain their weight, for example following a period of weight loss intervention.

In particular, the agents of the invention may support weight maintenance through appetite suppression or control during and/or following a period of weight loss intervention (e.g. a diet or exercise regime).

In one aspect, the invention provides the non-therapeutic use of an agent of the invention to maintain a healthy body composition, for example after a period of weight loss.

### Obesity

“Overweight” is defined for an adult human as having a body mass index (BMI) between 25 and 30.

“Body mass index” means the ratio of weight in kg divided by the height in metres, squared.

“Obesity” is a condition in which the natural energy reserve, stored in the fatty tissue of animals, in particular humans and other mammals, is increased to a point where it is associated with certain health conditions or increased mortality. “Obese” is defined for an adult human as having a BMI greater than 30.

“Normal weight” is defined for an adult human as having a BMI of 18.5 to 25, whereas “underweight” may be defined as a BMI of less than 18.5.

Obesity is a chronic metabolic disorder that has reached epidemic proportions in many areas of the world and is the major risk factor for serious co-morbidities such as type 2 diabetes mellitus, cardiovascular disease, dyslipidaemia and certain types of cancer (World Health Organ. Tech. Rep. Ser. (2000) 894: i-xii, 1-253).

“Obesity-related disorder” refers to any condition which an obese individual is at an increased risk of developing. Obesity-related disorders include diabetes (e.g. type 2 diabetes), stroke, high cholesterol, cardiovascular disease, insulin resistance, coronary heart disease, metabolic syndrome, hypertension and fatty liver.

### Methods of Screening

The invention provides agents that are capable of increasing the activity of BCKDH and/or decreasing the activity of BCKDH kinase, and additionally provides methods for identifying such agents.

The agents of the invention may be identified by methods that provide either qualitative or quantitative results. Furthermore, such methods may be used to characterise as well as identify agents of the invention.

The candidate agents may be any agents of potential interest, for example peptides, polypeptides (e.g. antibodies), nucleic acids or small molecules. Preferably, the candidate agents are compounds or mixtures of potential therapeutic interest. Preferably, the candidate agents are of low toxicity for mammals, in particular humans. In some embodiments, the candidate agents may comprise nutritional agents and/or food ingredients, including naturally-occurring compounds or mixtures of compounds such as plant or animal extracts.

The candidate agents may form part of a library of agents, for example a library produced by combinatorial chemistry or a phage display library. In one embodiment, the candidate agents form part of a library of plant bioactive molecules.

### BCKDH Activity

The invention also provides methods for identifying agents that are capable of increasing the activity of BCKDH and agents that are identified by such methods. The activity of BCKDH may be analysed directly, for example by analysing the enzymatic activity of the BCKDH.

A number of techniques are known in the art for measuring BCKDH activity. These techniques may be applied to BCKDH that has been isolated from a cell. The BCKDH may have been expressed using recombinant techniques. Preferably, the BCKDH has been purified.

In one embodiment, BCKDH is determined spectrophotometrically by monitoring the production of NADH from NAD⁺, for example in the presence of α-ketoisovaleric acid, a substrate for BCKDH. Such assay techniques are described in, for example, Hawes, J. W. et al. (2000) Methods Enzymol. 324: 200-207.

### BCKDH Kinase Activity

The invention also provides methods for identifying agents that are capable of decreasing the activity of BCKDH kinase and agents that are identified by such methods. The activity of BCKDH kinase may be analysed directly, for example by analysing the enzymatic activity of the BCKDH kinase.

The ability of a candidate agent to reduce the activity of a protein, for example an enzyme such as a kinase, may be expressed in terms of an IC50 value. The IC50 is the concentration of an agent that is required to give rise to a 50% reduction in the activity of the protein (e.g. a 50% reduction in enzymatic activity). The calculation of IC50 values is well known in the art. Preferably, the agents of the invention have an IC50 value for inhibition of BCKDH kinase of less than 100 μM, more preferably less than 10 μM, for example less than 1 μM, less than 100 nM or less than 10 nM.

A number of techniques are known in the art for measuring kinase activity. These techniques may be applied to a kinase, for example BCKDH kinase, that has been isolated from a cell. The BCKDH kinase may have been expressed using recombinant techniques. Preferably, the BCKDH kinase has been purified.

In one embodiment, kinase activity is determined by monitoring the incorporation of phosphate into a substrate, for example radiolabelled phosphate from [γ-³²P]-labelled ATP into a BCKDH substrate or suitable fragment thereof. Such assay techniques are described in, for example, Hastie, C. J. et al. (2006) Nat. Protocols 1: 968-971.

In another embodiment, kinase activity is determined by monitoring the amount of ADP that is produced in a kinase reaction (e.g. monitoring the rate of ADP production). Such assay systems (such as the commercial ADP-Glo™ Kinase Assay produced by Promega) may be based on the reconversion of ADP (produced in the kinase reaction) to ATP, which may be detected, for example via the production of a luminescent signal by a luciferase. In such an assay, the luminescent signal correlates with kinase activity. Such assays are particularly suitable for determining the effects of candidate agents on the activity of a broad range of purified kinases and are well suited to use in high-throughput screening.

In another embodiment, kinase activity is determined by monitoring the amount of ATP that remains at certain time points during a reaction (e.g. monitoring the rate of ATP consumption). In such assays, the signal correlates with the amount of ATP present, which inversely correlates with the kinase activity. Such assay systems (such as the commercial Kinase-Glo® Kinase Assay by Promega) may be based on the production of a luminescent signal by a luciferase.

### BCKDH and BCKDH Kinase Binding

The invention also provides methods of identifying agents which are capable of binding to BCKDH (in particular BCKDH E1 B subunit) and/or BCKDH kinase and, alternatively or additionally, characterising such binding. For example, the method may allow measurement of absolute or relative binding affinity, and/or enthalpy and entropy of binding. Binding affinity may be expressed in terms of the equilibrium dissociation (K_{d}) or association (Kₐ) constant.

A number of assay techniques are known in the art for identifying binding between a candidate agent and a protein. The assay technique employed is preferably one which is amenable to automation and/or high throughput screening of candidate agents. The assay may be performed on a disposable solid support such as a microtitre plate, microbead, resin or similar.

For example, target BCKDH (in particular BCKDH E1 B subunit) and/or BCKDH kinase may be immobilised on a solid support, for example a microbead, resin, micotitre plate or array. Candidate agents may then be contacted with the immobilised target protein. Optionally, a wash procedure may be applied to remove weakly or non-specifically binding agents. Any agents binding to the target protein may then be detected and identified. To facilitate the detection of bound agents, the candidate agents may be labelled with a readily detectable marker. The marker may comprise, for example, a radio label, an enzyme label, an antibody label, a fluorescent label, a particulate (e.g. latex or gold) label or similar.

Alternatively, the above procedure may be reversed and the candidate agents may be immobilised and the target BCKDH (in particular BCKDH E1 B subunit) and/or BCKDH kinase may be contacted with said immobilised agents. Optionally, a wash procedure may be applied to remove weakly or non-specifically bound target protein. Any agents to which BCKDH (in particular BCKDH E1 B subunit) and/or BCKDH kinase the binds may then be detected and identified. To facilitate the detection of binding, the BCKDH (in particular BCKDH E1 B subunit) and/or BCKDH kinase may be labelled with a readily detectable marker as described above.

In addition to the assays described above, other suitable assay techniques are known in the art. Examples of such techniques include radioassays, fluorescence assays, ELISA, fluorescence polarisation, fluorescence anisotropy, isothermal titration calorimetry (ITC), surface plasmon resonance (SPR) and the like. These assays may be applied to identify agents which bind to BCKDH (in particular BCKDH E1 B subunit) and/or BCKDH kinase. Indeed, platforms for the automation of many of these techniques are widely known in the art to facilitate high-throughput screening.

More than one assay techniques may be used to provide a detailed understanding of a candidate agent's binding to BCKDH (in particular BCKDH E1 B subunit) and/or BCKDH kinase. For example, assays which provide qualitative binding information may be used as a first step in the method, followed by further assays using different techniques to provide quantitative binding data and/or data on the effect on activity of the target protein.

The assay techniques described above may be adapted to perform competition binding studies. For example, these techniques are equally suitable to analyse the binding of a protein to substrate or cofactor in the presence of a candidate agent. It will therefore be possible to use the above techniques to screen and identify agents that modulate the binding between a protein and its substrate or cofactor, thus having an effect on the protein's activity. For example, these assays will enable the detection of binding between the BCKDH E1 subunit and thiamine diphosphate, or between BCKDH kinase and ATP, in the presence of a candidate agent.

Preferably, the agents of the invention will bind with high affinity. For example, the agents of the invention will bind to BCKDH (in particular BCKDH E1 B subunit) and/or BCKDH kinase with a K_{d} of less than 100 μM, more preferably less than 10 μM, for example less than 1 μM, less than 100 nM or less than 10 nM.

Binding affinity may be measured using standard techniques known in the art, e.g. surface plasmon resonance, ELISA and so on (for instance as described above), and may be quantified in terms of either dissociation (K_{d}) or association (Kₐ) constants.

Bioinformatics-based approaches, such as in silico structure-guided screening, may also be used to identify agents of the invention.

### BCKDH and BCKDH Kinase Levels

The invention provides agents for increasing BCKDH (in particular BCKDH E1 B subunit) levels and/or decreasing BCKDH kinase levels. Levels of the relevant protein may be equated with levels of expression of the protein in a cell or organism. Protein levels may be analysed directly or indirectly, for example by analysis of levels of mRNA encoding the protein.

Methods for analysing the expression of BCKDH (in particular BCKDH E1 B subunit) and/or BCKDH kinase may be employed in the invention to screen the effect of a candidate agent on the protein's levels.

A number of techniques are known in the art for determining the expression level of a protein. These techniques may be applied to test the effect of candidate agents on the expression level of BCKDH (in particular BCKDH E1 B subunit) and/or BCKDH kinase. The technique employed is preferably one which is amenable to automation and/or high throughput screening of candidate agents.

For example, screens may be carried out using cells harbouring polynucleotides encoding BCKDH (in particular BCKDH E1 B subunit) and/or BCKDH kinase operably linked to a reporter moiety. The reporter moiety may be operably linked to endogenous BCKDH—(in particular BCKDH E1 B subunit) and/or BCKDH kinase-encoding genes. Alternatively, exogenous copies of BCKDH (in particular BCKDH E1 B subunit) and/or BCKDH kinase operably linked to a reporter moiety may be inserted into a cell. In this embodiment, the cell may be engineered to be deficient for natural BCKDH and/or BCKDH kinase expression. The reporter moieties linked to BCKDH and/or BCKDH kinase may be different and distinguishable from one another. Suitable reporter moieties include fluorescent labels, for example fluorescent proteins such as green, yellow, cherry, cyan or orange fluorescent proteins.

By “operably linked” it is to be understood that the components described are in a relationship permitting them to function in their intended manner.

Such cells may be contacted with candidate agents and the level of expression of BCKDH (in particular BCKDH E1 B subunit) and/or BCKDH kinase may be monitored by analysing the level of reporter moiety expression in the cell. Fluorescent reporter moieties may be analysed by a number of techniques known in the art, for example flow cytometry, fluorescence-activated cell sorting (FACS) and fluorescence microscopy. Expression of BCKDH (in particular BCKDH E1 B subunit) and/or BCKDH kinase may be analysed separately or simultaneously within the same cell. Expression levels of BCKDH (in particular BCKDH E1 B subunit) and/or BCKDH kinase may be compared before and after contact with the candidate agent. Alternatively, expression levels of BCKDH (in particular BCKDH E1 B subunit) and/or BCKDH kinase may be compared between cells contacted with a candidate agent and control cells.

Other methods may be used for analysing the expression of proteins, for example BCKDH (in particular BCKDH E1 B subunit) and/or BCKDH kinase. Protein expression may be analysed directly. For example, expression may be quantitatively analysed using methods such as SDS-PAGE analysis with visualisation by Coomassie or silver staining. Alternatively expression may be quantitatively analysed using Western blotting or enzyme-linked immunosorbent assays (ELISA) with antibody probes which bind the protein product. BCKDH (in particular BCKDH E1 B subunit) and/or BCKDH kinase labelled with reporter moieties, as described above, may also be used in these methods. Alternatively, protein expression may be analysed indirectly, for example by studying the amount of mRNA corresponding to the protein that is transcribed in a cell. This can be achieved using methods such as quantitative reverse transcription PCR and Northern blotting.

Similar techniques may also be used for the analysis of leptin protein expression.

### Agents

The invention provides agents that are capable of increasing the activity of BCKDH and/or decreasing the activity of BCKDH kinase, and additionally provides methods for identifying such agents.

The agents of the invention may be, for example, peptides, polypeptides (e.g. antibodies), nucleic acids (e.g. siRNAs, shRNAs, miRNAs and antisense RNAs) or small molecules. Preferably, the agents are of low toxicity for mammals, in particular humans. In some embodiments, the agents may comprise nutritional agents and/or food ingredients, including naturally-occurring compounds or mixtures of compounds such as plant or animal extracts.

In one embodiment, the agent of the invention is resveratrol. Resveratrol (3,5,4′-trihydroxy-trans-stilbene) is a stilbenoid and phytoalexin that is naturally produced by a number of plants in response to injury or when under attack by pathogens. Food sources of resveratrol include the grape skins, blueberries, raspberries and mulberries.

In one embodiment, the agent of the invention is valproic acid. Valproic acid is a medicament used in the treatment of epilepsy, bipolar disorder and migraines. It may prevent seizures in subjects with absence seizures, partial seizures and generalised seizures. The structure of valproic acid is:

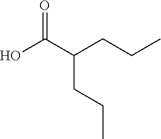

In one embodiment, the agent of the invention is α-chloroisocaproic acid. α-Chloroisocaproic acid is an analogue of leucine and is the most potent known inhibitor of BCKDH kinase (Skimomura, Y. et al. (2006) J. Nutr. 136: 250S-253S). The structure of α-chloroisocaproic acid is:

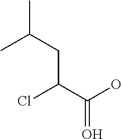

In one embodiment, the agent of the invention is α-ketoisocaproic acid (KIC). α-Ketoisocaproic acid, the transamination product of leucine is a known physiological inhibitor of BCKDH kinase (Shimomura, Y. et al. (2006) J. Nutr. 136: 250S-253S). The structure of α-ketoisocaproic acid is:

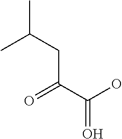

Example agents that affect the activity of BCKDH particularly through affecting the activity of the BCKDH E1 B subunit include the agents recited in Table 1a (Davis A P, et al. The Comparative Toxicogenomics Database: update 2017. Nucleic Acids Res. 2016 Sep. 19).

TABLE 1a
Agents that increase the activity of branched-chain
alpha-keto acid dehydrogenase E1 B subunit (BCKDHB).

| | Chemical | | |
|---|---|---|---|
| Chemical Name | ID | CAS RN | Interaction Actions |
| 2,4-dinitrotoluene | C016403 | 121-14-2 | affects expression |
| Ammonium Chloride | D000643 | 12125-02-9 | affects expression |
| Antirheumatic Agents | D018501 | | increases expression |
| Benzo(a)pyrene | D001564 | 50-32-8 | affects expression/ |
| | | | affects reaction |
| Benzo(a)pyrene | D001564 | 50-32-8 | increases expression |
| Cuprizone | D003471 | 370-81-0 | increases expression |
| Diethylnitrosamine | D004052 | 55-18-5 | increases expression |
| Methylmercuric chloride | C004925 | 115-09-3 | increases expression |
| pirinixic acid | C006253 | 50892-23-4 | increases expression |
| potassium chromate(VI) | C027373 | 7789-00-6 | increases expression |
| Tetrachlorodibenzodioxin | D013749 | 1746-01-6 | affects expression |
| Valproic Acid | D014635 | 99-66-1 | affects expression |
| Valproic Acid | D014635 | 99-66-1 | increases expression |
| Vancomycin | D014640 | 1404-90-6 | increases expression |

Example agents that affect the activity of BCKDH particularly through affecting the activity of the BCKDH E1 A subunit include the agents recited in Table 1b (Davis A P, et al. The Comparative Toxicogenomics Database: update 2017. Nucleic Acids Res. 2016 Sep. 19).

TABLE 1b
Agents that increase the activity of branched-chain
alpha-keto acid dehydrogenase E1 A subunit (BCKDHA).

| | Chemical | | |
|---|---|---|---|
| Chemical Name | ID | CAS RN | Interaction Actions |
| 1,12-benzoperylene | C006718 | 191-24-2 | increases expression |
| 17-ethynyl-5-androstene- | C524733 | | affects binding |
| 3,7,17-triol | | | |
| 2,4-dinitrotoluene | C016403 | 121-14-2 | affects expression |
| Acetaminophen | D000082 | 103-90-2 | affects expression |
| Acetaminophen | D000082 | 103-90-2 | increases expression |
| Amiodarone | D000638 | 1951-25-3 | increases expression |
| Ammonium Chloride | D000643 | 12125-02-9 | affects expression |
| Atrazine | D001280 | 1912-24-9 | increases expression |
| Bisphenol A | C006780 | 80-05-7 | increases expression |
| Carbamazepine | D002220 | 298-46-4 | affects expression |
| Carbon Tetrachloride | D002251 | 56-23-5 | increases expression |
| Chloroprene | D002737 | 126-99-8 | increases expression |
| Clofibrate | D002994 | 637-07-0 | increases expression |
| Ethinyl Estradiol | D004997 | 57-63-6 | increases expression |
| Ethinyl Estradiol | D004997 | 57-63-6 | affects cotreatment/ |
| | | | increases expression |
| Fluorouracil | D005472 | 51-21-8 | affects expression |
| Furan | C039281 | 110-00-9 | affects binding |
| Ketamine | D007649 | 6740-88-1 | increases expression |
| Methylmercuric chloride | C004925 | 115-09-3 | increases expression |
| Pirinixic acid | C006253 | 50892-23-4 | increases expression |
| Streptozocin | D013311 | 18883-66-4 | affects expression |
| Tetrachlorodibenzodioxin | D013749 | 1746-01-6 | affects expression |
| Tetrachlorodibenzodioxin | D013749 | 1746-01-6 | affects cotreatment/ |
| | | | increases expression |
| Tetrachlorodibenzodioxin | D013749 | 1746-01-6 | increases expression |
| Tetracycline | D013752 | 60-54-8 | increases expression |
| Topotecan | D019772 | 123948-87-8 | affects response |
| | | | to substance |
| Tunicamycin | D014415 | 11089-65-9 | increases expression |
| Valproic Acid | D014635 | 99-66-1 | affects expression |
| Vancomycin | D014640 | 1404-90-6 | increases expression |
| Vinclozolin | C025643 | 50471-44-8 | increases expression |

Example agents that affect the activity of BCKDH kinase include the agents recited in Table 2 (Davis A P, et al. The Comparative Toxicogenomics Database: update 2017. Nucleic Acids Res. 2016 Sep. 19).

TABLE 2
Agents that decrease the activity of branched-chain
alpha-keto acid dehydrogenase kinase (BCKDH kinase).

| | Chemical | | |
|---|---|---|---|
| Chemical Name | ID | CAS RN | Interaction Actions |
| Acetaminophen | D000082 | 103-90-2 | affects expression |
| Ammonium Chloride | D000643 | 12125-02-9 | affects expression |
| Arbutin | D001104 | 497-76-7 | decreases expression |
| Atrazine | D001280 | 1912-24-9 | decreases expression |
| Bisphenol A | C006780 | 80-05-7 | affects expression |
| Cacodylic Acid | D002101 | 75-60-5 | decreases expression |
| Clofibrate | D002994 | 637-07-0 | decreases expression |
| Clofibric Acid | D002995 | 882-09-7 | affects cotreatment/ |
| | | | affects expression |
| Cobaltous chloride | C018021 | 7646-79-9 | decreases expression |
| Copper | D003300 | 7440-50-8 | affects binding/ |
| | | | decreases expression |
| Copper Sulfate | D019327 | 7758-98-7 | decreases expression |
| Dibutyl Phthalate | D003993 | 84-74-2 | decreases expression |
| Diethylnitrosamine | D004052 | 55-18-5 | affects cotreatment/ |
| | | | affects expression |
| Formaldehyde | D005557 | 50-00-0 | decreases expression |
| Hydrogen Peroxide | D006861 | 7722-84-1 | affects expression |
| Hypochlorous Acid | D006997 | 7790-92-3 | decreases expression |
| Ketolides | D048628 | | decreases expression |
| Methoxyacetic acid | C013598 | 625-45-6 | affects expression |
| NSC 689534 | C558013 | | affects binding/ |
| | | | decreases expression |
| Ochratoxin A | C025589 | 303-47-9 | decreases expression |
| Procymidone | C035988 | 32809-16-8 | decreases expression |
| Sodium bichromate | C016104 | 10588-01-9 | decreases expression |
| Tetrachlorodibenzodioxin | D013749 | 1746-01-6 | affects reaction/ |
| | | | decreases expression |
| Tetrachlorodibenzodioxin | D013749 | 1746-01-6 | affects expression |
| Tetrachlorodibenzodioxin | D013749 | 1746-01-6 | decreases expression |
| Thapsigargin | D019284 | 67526-95-8 | decreases expression |
| Tunicamycin | D014415 | 11089-65-9 | decreases expression |
| Valproic Acid | D014635 | 99-66-1 | affects expression |

The agents for use according to the invention may be, for example, present as salts or esters, in particular pharmaceutically acceptable salts or esters.

siRNAs, shRNAs, miRNAs and Antisense DNAs/RNAs

Expression of BCKDH kinase may be modulated using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11: 429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA>30 bp has been found to activate the interferon response leading to shut-down of protein synthesis and non-specific mRNA degradation (Stark et al. (1998) Ann. Rev. Biochem. 67: 227-64). However, this response can be bypassed by using 21 nt siRNA duplexes (Elbashir et al. (2001) EMBO J. 20: 6877-88; Hutvagner et al. (2001) Science 293: 834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) noncoding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art. Furthermore, methods for specifically modulating (e.g. reducing) expression of a protein in a certain cell type within an organism, for example through the use of tissue-specific promoters are well known in the art.

### Antibodies

The term “antibody”, as used herein, refers to complete antibodies or antibody fragments capable of binding to a selected target, and includes Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

Methods for the production of antibodies are known by the skilled person. Alternatively, antibodies may be derived from commercial sources.

If polyclonal antibodies are desired, a selected mammal (e.g. mouse, rabbit, goat or horse) may be immunised. Serum from the immunised animal may be collected and treated according to known procedures. If the serum contains polyclonal antibodies to other antigens, the polyclonal antibodies may be purified by immunoaffinity chromatography. Techniques for producing and processing polyclonal antisera are known in the art.

Monoclonal antibodies directed against antigens (e.g. proteins) used in the invention can also be readily produced by the skilled person. The general methodology for making monoclonal antibodies by hybridomas is well known. Immortal antibody-producing cell lines can be created by cell fusion and also by other techniques such as direct transformation of B-lymphocytes with oncogenic DNA or transfection with Epstein-Barr virus. Panels of monoclonal antibodies produced against antigens can be screened for various properties, for example for isotype and epitope affinity.

An alternative technique involves screening phage display libraries where, for example, the phage express scFv fragments on the surface of their coat with a large variety of complementarity determining regions (CDRs). This technique is well known in the art.

Antibodies, both monoclonal and polyclonal, which are directed against antigens, are particularly useful in diagnosis, and those which are neutralising are useful in passive immunotherapy. Monoclonal antibodies in particular may be used to raise anti-idiotype antibodies. Anti-idiotype antibodies are immunoglobulins which carry an “internal image” of the antigen of the infectious agent against which protection is desired.

Techniques for raising anti-idiotype antibodies are known in the art. These anti-idiotype antibodies may also be useful for treatment, as well as for an elucidation of the immunogenic regions of antigens.

Introduction of Polypeptides and Polynucleotides into Cells

An agent for use in the invention may be, for example, a polypeptide or a polynucleotide. Polynucleotides and polypeptides may also need to be introduced into cells as part of the methods or screening assays of the invention.

Where the invention makes use of a polypeptide, the polypeptides may be administered directly to a cell (e.g. the polypeptide itself may be administered), or by introducing polynucleotides encoding the polypeptide into cells under conditions that allow for expression of the polypeptide in a cell of interest. Polynucleotides may be introduced into cells using vectors.

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred to a target cell. The vector may serve the purpose of maintaining the heterologous nucleic acid (e.g. DNA or RNA) within the cell, facilitating the replication of the vector comprising a segment of nucleic acid or facilitating the expression of the protein encoded by a segment of nucleic acid. Vectors may be non-viral or viral. Examples of vectors used in recombinant nucleic acid techniques include, but are not limited to, plasmids, chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid or virus vectors and may include a promoter for the expression of a polynucleotide and optionally a regulator of the promoter.

Vectors comprising polynucleotides used in the invention may be introduced into cells using a variety of techniques known in the art, such as transduction and transfection. Several techniques suitable for this purpose are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell.

Transfer of the polypeptide or polynucleotide may be performed by any of the methods known in the art which may physically or chemically permeabilise the cell membrane. Cell-penetrating peptides may also be used to transfer a polypeptide into a cell.

In addition, the invention may employ gene targeting protocols, for example the delivery of DNA-modifying agents.

The vector may be an expression vector. Expression vectors as described herein comprise regions of nucleic acid containing sequences capable of being transcribed. Thus, sequences encoding mRNA, tRNA and rRNA are included within this definition.

Expression vectors preferably comprise a polynucleotide for use in the invention operably linked to a control sequence that is capable of providing for the expression of the coding sequence by the host cell. A regulatory sequence “operably linked” to a coding sequence is ligated in such a way that expression of the coding sequence is achieved under conditions compatible with the control sequence. The control sequence may be modified, for example by the addition of further transcriptional regulatory elements to make the level of transcription directed by the control sequence more responsive to transcriptional modulators.

### Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides, such as DNA polynucleotides, may be produced recombinantly, synthetically or by any means available to the skilled person. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA, for example mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Proteins

As used herein, the term “protein” includes single chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds.

### Variants, Derivatives, Analogues, Homologues and Fragments

In addition to the specific proteins and nucleotides mentioned herein, the present invention also encompasses variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein, in relation to proteins or polypeptides of the invention, includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein, in relation to polypeptides or polynucleotides, includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table 3 below. Amino acids in the same block in the second column and preferably in the same line in the third column may be substituted for each other:

TABLE 3
Conservative substitutions of Amino Acids

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” means an entity having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60).

However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

### Method of Treatment

It is to be appreciated that all references herein to treatment include curative, palliative and prophylactic treatment. The treatment of mammals, particularly humans, is preferred. Both human and veterinary treatments are within the scope of the present invention.

### Administration

Although the agents for use in the invention can be administered alone, they will generally be administered in admixture with a pharmaceutical carrier, excipient or diluent, particularly for human therapy.

In some embodiments, the agent is a nutritional agent, food additive or food ingredient, and may thus be formulated in a suitable food composition. Thus, the agent may be administered, for example, in the form of a food product, drink, pet food product, food supplement, nutraceutical or nutritional formula.

### Dosage

The skilled person can readily determine an appropriate dose of one of the agents of the invention to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

A “subject” refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

### EXAMPLES

### Example 1

This study relates to a protein quantitative trait loci (pQTL) analysis performed on Diogenes weight loss intervention data. The Diogenes study is a pan-European, randomised and controlled dietary intervention study investigating the effects of dietary protein and glycaemic index on weight loss and weight maintenance in obese and overweight families in eight European centres (Larsen et al. (2009) Obesity Rev. 11: 76-91). In brief, the Diogenes study subjected screened participants to a low-calorie diet (LCD) phase (CID1), in which the overweight/obese subjects followed an 8 week Modifast® diet (approximately 800 kCal/day), followed by a weight maintenance phase (CID2).

This is the first study that has tested the association between common variants genotyped on an Illumina chip and proteins expression change during intervention focusing on proteins associated to body mass index (BMI) change.

In this study, SNPs not observed on the Illumina chip were imputed using the Minimac3 tool using the European 1000 Genome population as the reference genome. Analysis was performed using best-guess genotypes (calculated on the basis of the 80% best-guess genotypes) from variants passing specific QC thresholds: MAF≥0.01 and significant deviation from Hardy-Weinberg equilibrium (P>1.0e−6). Based on these new data, a new pQTL analysis was performed to extract more association signals in previously associated regions or identify new regions.

### Materials and Methods

### Data

The cohort included 498 participants with information at CID1 and CID2 for 1129 Somalogic proteins extracted from plasma. Genetic data imputation led to 4020756 SNPs that passed QC processes. Protein expression information was obtained using Somalogic technology. Data were pre-processed and controlled for quality. Gene expression (rnaSEQ technology, information available for almost 15000 transcripts from adipose tissue after quality control) and metabolomics data were also available and used this study.

### Methods

Association between BMI and each protein expression change during the low-calorie diet (LCD) was tested using a linear regression (“univariate” regression). BMI change was first regressed on confounding cofactors (sex, age and centre), and residuals were regressed against delta protein expression. P-values were corrected for multiple testing using Benjamini-Hochberg standard false discovery rate correction.

pQTL association between SNPs and protein expression was performed using a linear mixed model (LMM). LMM is an emerging method of choice for association mapping, which allows for correction of genetic population heterogeneity (geographic population structure generated by the different recruitment centres across Europe). The basic approach is to build a genetic relationship matrix (GRM) modelling genome-wide sample structure (the genetic background of all patients in the study) using all (or part of the) SNPs available on the chip (imputed SNPs were not used during this step). Its contribution to protein expression variance is estimated using a random-effects model and association statistic is computed accounting for this component of variance. LMM association methods are effective in preventing false-positive associations between genetic variants and traits in studies of human and model organisms. In the present study, the dependent variable was the protein expression residuals from regression on age, sex and centre, and the independent variables were the SNPs. For each protein, a genome-wide association study (GWAS) was performed testing each SNP independently.

GCTA software was used for LMM computation with the “loco” option that excludes all SNPs belonging to the same chromosome than the SNP under study to avoid multi-collinearity. If the SNP under study, and all SNPs in linkage disequilibrium were used in the GRM, the log likelihood of the null model would be higher than it should be and lead to deflation of the test statistic and loss of power. This phenomenon is called “proximal contamination”.

GWASs pQTL were performed for all proteins. Results were extracted for proteins with delta expression associated to BMI change during intervention. No correction for multiple testing was applied. Our objective was to highlight/prioritise pQTL for further analysis using other omics information including transcriptomics and genetics (genome sequencing).

Results were plotted using locusZoom software implemented in a R script (launch_locuszoom.R) using 1000 Genomes European genetic data as reference (hg19).

Gene co-expression was evaluated using GeneMANIA (Zuberi, K. et al. (2013) Nucleic Acids Res. 41(Web Server issue): W115-22). By definition, two genes are linked (co-expressed) if their expression levels are similar across conditions in a gene expression study. Most of these data are collected from the Gene Expression Omnibus (GEO) and only data associated with a publication are collected.

A pipeline was written in R from Minimac imputed data output to results extraction including QC steps, parallelised pQTL GWASs, extraction of significant signals and plots.

Based on a set of “top” proteins for which change in expression during the LCD was associated to BMI change, pQTL results were extracted and investigated. Change in protein or expression, BMI or other covariates during the LCD implies change in expression/level before and after intervention unless specified.

### Results

Before starting the analysis, for all proteins, SNPs with a FDR q-value<0.20 and MAF>0.05 were extracted. Their cis/trans acting effect was then evaluated according to their position +/−500kb around the corresponding coding gene. A q-value measures the False Discovery Rate (FDR) incurred by accepting the given test and every test with a smaller p-value (and maybe even larger p-values, if they improve the FDR).

No cis-acting SNP reached a q-value <0.05 association cut-off for all 1129 proteins. Relaxing FDR cutoff to 0.20 did not identify any cis-acting SNPs, only trans-acting.

### Protein Expression Change During LCD

After correction for multiple testing and assuming a p-value cutoff set to 5%, 55 proteins were positively and 52 negatively correlated to BMI during weight loss intervention. A correlation heat-map was built based on Kendal correlation tau correlation coefficient for all proteins associated to BMI during LCD Kendall tau rank correlation is a non-parametric test for statistical dependence between two ordinal (or rank-transformed) variables (similar to Spearman's), but which, unlike Spearman's, can handle ties. Hierarchical clustering was used to identify potential clusters. We did not observe clearly delineated large blocks of proteins possibly because of numerous false positive results in this large list.

Association with P<1.0e−06 (after BH multiple testing correction) was observed for 9 proteins. A block of very significant correlation was observed between these 9 proteins the Kendall correlation level for protein pairs with p-value corrected for multiple testing under 5% after Bonferroni correction. Two anti-correlated blocks of proteins were observed including all but IL1 RAP gene coding-protein.

Table 4 provides an overview of these 9 proteins with Somalogic ID, name of coding gene, UNIPROT ID, direction of correlation with BMI and p-value (estimated p-value, PVAL; and multiple testing corrected, p-value, PBH, based on the Benjamin-Hochberg method (BH)). A first block of proteins including leptin, growth-hormone receptor, TIG2 (chemerin) and SAP was positively correlated with BMI change during the LCD while a second block including NRP1, SHBG, IGFBP-2, angiopoietin-2 and IL-1 R AcP was negatively correlated to BMI change.

TABLE 4
Top proteins associated to BMI change during the LCD.

| TARGET | GENE | UNIPROT | Correlation | PVAL | PBH |
|---|---|---|---|---|---|
| Growth | GHR | P10912 | positive | 5.2e−26 | 5.2e−26 |
| hormone | | | | | |
| receptor | | | | | |
| Leptin | LEP | P41159 | positive | 3.9e−21 | 3.9e−21 |
| SHBG | SHBG | P04278 | negative | 1.1e−16 | 1.1e−16 |
| IGFBP-2 | IGFBP2 | P18065 | negative | 2.8e−16 | 2.8e−16 |
| NRP1 | NRP1 | O14786 | negative | 8.5e−13 | 8.5e−13 |
| TIG2 | RARRES2 | Q99969 | positive | 4.9e−12 | 4.9e−12 |
| Angiopoietin-2 | ANGPT2 | O15123 | negative | 8.5e−12 | 8.5e−12 |
| IL-1 R AcP | IL1RAP | Q9NPH3 | negative | 1.3e−11 | 1.3e−11 |
| SAP | APCS | P02743 | positive | 1.3e−09 | 1.3e−09 |

The results below relate to proteins with promising pQTL results in genes likely involved in obesity and/or related traits. Other genes were discarded as not having GWA pQTL enriched signals and top SNPs not targeting genes of potential interest.

### Leptin

pQTL Results

Leptin, the “satiety hormone”, is a hormone made by adipose cells that helps to regulate energy balance by inhibiting hunger. In obesity, a decreased sensitivity to leptin occurs, resulting in an inability to detect satiety despite high energy stores. Leptin levels fall during weight loss and increased brain activity occurs in areas involved in emotional, cognitive and sensory control of food intake. Restoration of leptin levels maintains weight loss and reverses the changes in brain activity. Thus, leptin is a critical factor linking reduced energy stores to eating behaviour (Ahima, R. S. (2008) J. Clin. Invest. 118: 2380-2383).

A QQ plot demonstrated the enrichment of association signal (genomic inflation factor (GIF)=1.0147153, 1.6930767×10⁻⁵). This enrichment targets a specific region on chromosome 6

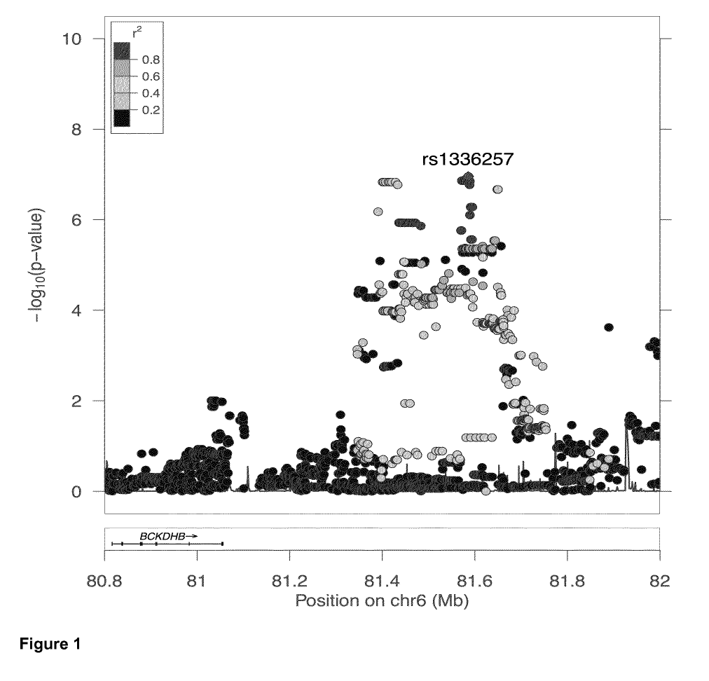

The top ten pQTL results for leptin includes SNPs in the same targeted region (Table 5 displays only the top 10 SNPs not in complete linkage disequilibrium (LD)). This region contains ncRNAs and is located in the regulatory region of the BCKDHB gene (http://www.genecards.org/cgi-bin/carddisp.pl?gene=BCKDHB&keywords=BCKDHB) FIG. 1 shows a Manhattan plot zooming in on this specific region of chromosome 6.

The BCKDHB enzyme complex is responsible for one step in the normal breakdown of leucine, isoleucine and valine. These three amino acids are obtained from the diet and are present in many kinds of food, particularly protein-rich foods such as milk, meat and eggs.

TABLE 5
Top 10 pQTL results for leptin.

| SnpsID | Chr | bp | Freq | b | p |
|---|---|---|---|---|---|
| rs1336257 | 6 | 81585576 | 0.2381 | 0.2255 | 1.111e−07 |
| rs507451 | 6 | 81571792 | 0.2397 | 0.2223 | 1.377e−07 |
| rs481481 | 6 | 81586692 | 0.2386 | 0.2238 | 1.408e−07 |
| rs9344031 | 6 | 81400749 | 0.09607 | 0.3105 | 1.476e−07 |
| rs4443477 | 6 | 81588425 | 0.238 | 0.2225 | 1.69e−07 |
| rs115586175 | 6 | 81433261 | 0.09401 | 0.3112 | 1.694e−07 |
| rs1981174 | 6 | 81588713 | 0.2385 | 0.2227 | 1.698e−07 |
| rs534800 | 6 | 81648445 | 0.1353 | 0.266 | 2.149e−07 |
| rs475407 | 6 | 81591054 | 0.237 | 0.2147 | 5.274e−07 |
| rs16892128 | 6 | 81391023 | 0.09544 | 0.2948 | 6.665e−07 |

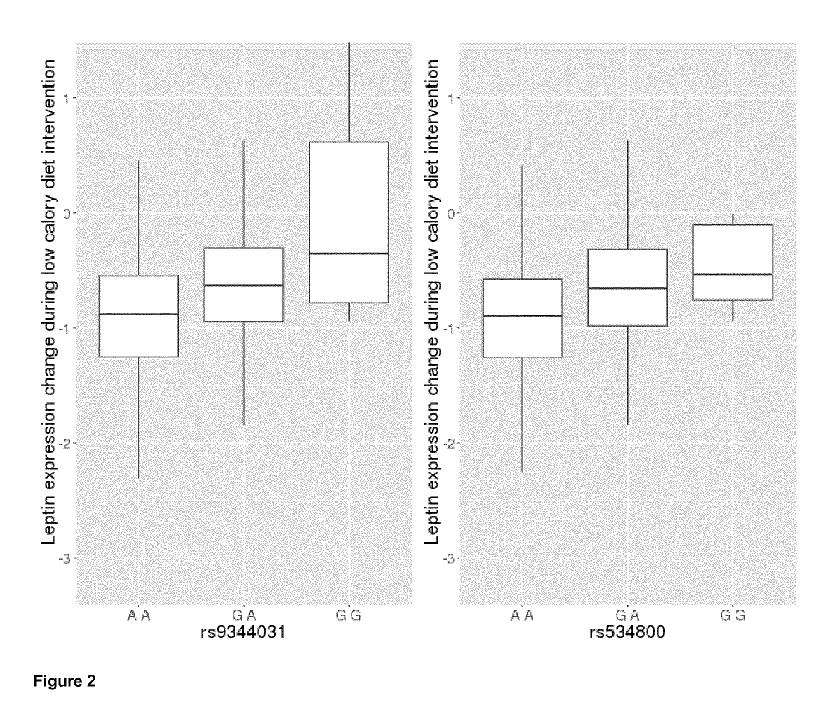

Protein expression stratified based on trans-acting SNP genotype did not underline a strong difference of expression despite significance shown in FIG. 2. However, since SNPs are generally surrogate markers of functional variant(s), it is likely that despite imputation the true underlying variant(s) is(are) not available.

### BCKDHB Gene Expression Analysis

Expression data from rnaSEQ was available for the BCKDHB gene. This gene was significantly down-regulated during LCD intervention (P=1.1e−11) after correction for multiple testing using Benjamini-Hochberg method. Association to BMI change was observed at the nominal level (P=0.016), but did not pass multiple testing correction (P=0.179). A significant and positive association was observed for a subset of 126 participants with proteomics and rnaSEQ data. BMI positively was associated (after correction for confounding cofactors) with leptin (P=1.6e−8) and BCKDHB (P=2.4e−2) expression, and leptin protein expression also displayed positive association with BCKDHB gene expression (P=2.6e−3).

Leptin gene and protein expression were highly correlated (association p−value=3.15e−9) like BCKDHB and leptin (LEP) change during LCD (p=2.8e−9) and to a lesser extent BCKDHB and leptin protein (p=0.0026). The direction of the association was the same (positive) for all paired of variable tested.

Integration with Metabolomics

The BCKD enzyme complex is active in mitochondria, where it is involved in the breakdown of leucine, isoleucine and valine to provide energy. Stroeve et al. (Stroeve, J. H. (2016) Obesity 24: 379-388) observed that valine contributed negatively to weight loss success.

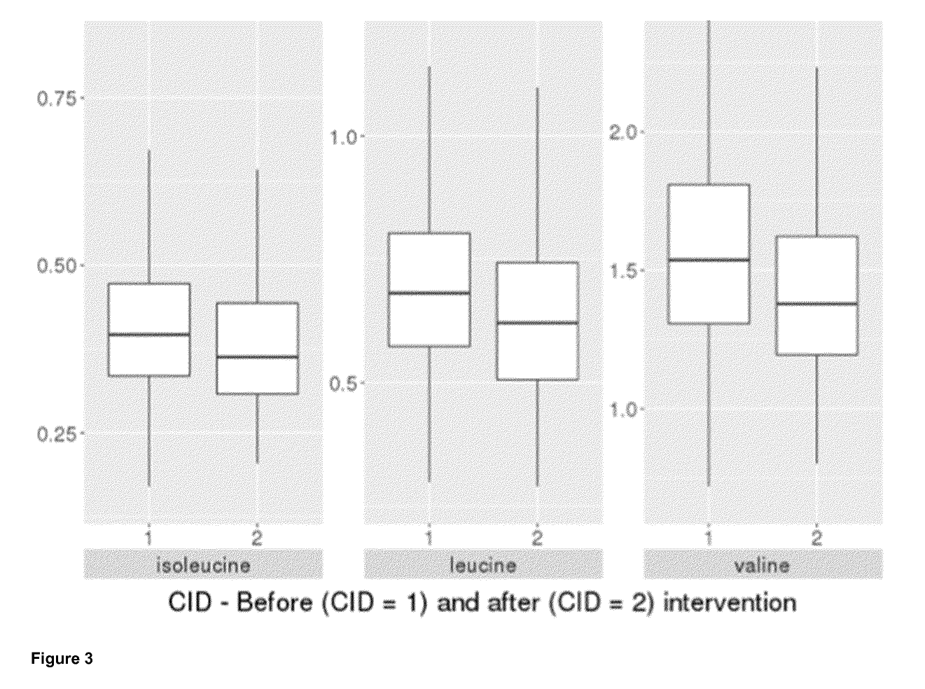

From an analysis of metabolomics data extracted for leucine, valine and isoleucine, all 3 branched-chain amino acids (BCAAs) were differentially expressed during the LCD (non parametric Wilcoxon paired test, Table 6 and FIG. 3).

TABLE 6
Wilcoxon paired test comparing BCAA before/after intervention.

| BCAA | P-value |
|---|---|
| valine | 2.5e−11 |
| isoleucine | 2.7e−07 |
| leucine | 1.5e−09 |

BCKDHB gene expression was associated to valine and leucine change during the LCD Table 7, but not isoleucine.

TABLE 7
Association between BCKDHB and metabolic parameters.

| BCAA | Estimate | Pr(>\|t\|) | P corrected |
|---|---|---|---|
| valine | 0.63 | 0.0091 | 0.013 |
| leucine | 0.65 | 0.0019 | 0.0058 |
| isoleucine | 0.22 | 0.22 | 0.22 |

All publications mentioned in the above specification are herein incorporated by reference.

Various modifications and variations of the described agents and methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1. Method for increasing leptin levels comprising administering an agent capable of increasing the activity of branched-chain alpha-keto acid dehydrogenase (BCKDH) to an individual in need of same.

2. Method according to claim 1 for use in supporting satiety.

3. Method according to claim 1, for use in supporting weight maintenance and/or treating or preventing obesity.

4. Method according to claim 1, wherein the agent increases the activity of the BCKDH E1 B subunit.

5. Method according to claim 1, wherein the agent is administered to a subject during or after a weight loss intervention.

6. Method according to claim 1, wherein the agent increases the level of BCKDH in a subject.

7. Method according to claim 1, wherein the agent does not affect the activity of branched-chain alpha-keto acid dehydrogenase kinase (BCKDH kinase).

8. Method according to claim 1, wherein the agent is selected from the group consisting of resveratrol and valproic acid.

9. Method according to claim 1, wherein the agent decreases the activity of branched-chain alpha-keto acid dehydrogenase kinase (BCKDH kinase).

10. Method according to claim 9, wherein the agent decreases the level of BCKDH kinase.

11. Method according to claim 9, wherein the agent is selected from the group consisting of α-chloroisocaproic acid and α-ketoisocaproic acid (KIC).

12. A method of identifying an agent capable of supporting weight maintenance and/or treating or preventing obesity in a subject comprising the steps:
(a) contacting a preparation comprising a branched-chain alpha-keto acid dehydrogenase (BCKDH) polypeptide or polynucleotide with a candidate agent; and
(b) detecting whether the candidate agent affects the activity of the BCKDH polypeptide or polynucleotide.

13. The method of claim 12, wherein the BCKDH is the BCKDH E1 B subunit.

14. The method of claim 12, wherein the method comprises contacting the preparation comprising BCKDH with a candidate agent and measuring the conversion of NAD+ to NADH.

15. A method of identifying an agent capable of supporting weight maintenance and/or treating or preventing obesity in a subject comprising the steps:
(a) contacting a preparation comprising a branched-chain alpha-keto acid dehydrogenase kinase (BCKDH kinase) polypeptide or polynucleotide with a candidate agent; and
(b) detecting whether the candidate agent affects the activity of the BCKDH kinase polypeptide or polynucleotide.

16. The method of claim 15, wherein the method comprises contacting the preparation comprising BCKDH kinase with a candidate agent in the presence of ATP and measuring the incorporation of phosphate into a substrate or measuring the conversion of ATP to ADP.


SEQUENCE LISTING (6 sequences)

SEQ ID NO: 1 - protein, 392 aa, Homo sapiens
  SIGNAL 1..50
MAVVAAAAGWLLRLRAAGAEGHWRRLPGAGLARGFLHPAATVEDAAQRRQVAHFTFQPDPEPREYGQTQKMNLFQSVTSALDNSLAKDPTAVIFGEDVAFGGVFRCTVGLRDKYGKDRVFNTPLCEQGIVGFGIGIAVTGATAIAEIQFADYIFPAFDQIVNEAAKYRYRSGDLFNCGSLTIRSPWGCVGHGALYHSQSPEAFFAHCPGIKVVIPRSPFQAKGLLLSCIEDKNPCIFFEPKILYRAAAEEVPIEPYNIPLSQAEVIQEGSDVTLVAWGTQVHVIREVASMAKEKLGVSCEVIDLRTIIPWDVDTICKSVIKTGRLLISHEAPLTGGFASEISSTVQEECFLNLEAPISRVCGYDTPFPHIFEPFYIPDKWKCYDALRKMINY

SEQ ID NO: 2 - DNA, 1179 bp, Homo sapiens
  source 1..1179: /mol_type=“unassigned DNA” /organism=“Homo sapiens”
atggcggttgtagcggcggctgccggctggctactcaggctcagggcggcaggggctgaggggcactggcgtcggcttcctggcgcggggctggcgcggggctttttgcaccccgccgcgactgtcgaggatgcggcccagaggcggcaggtggctcattttactttccagccagatccggagccccgggagtacgggcaaactcagaaaatgaatcttttccagtctgtaacaagtgccttggataactcattggccaaagatcctactgcagtaatatttggtgaagatgttgcctttggtggagtctttagatgcactgttggcttgcgagacaaatatggaaaagatagagtttttaataccccattgtgtgaacaaggaattgttggatttggaatcggaattgcggtcactggagctactgccattgcggaaattcagtttgcagattatattttccctgcatttgatcagattgttaatgaagctgccaagtatcgctatcgctctggggatctttttaactgtggaagcctcactatccggtccccttggggctgtgttggtcatggggctctctatcattctcagagtcctgaagcattttttgcccattgcccaggaatcaaggtggttatacccagaagccctttccaggccaaaggacttcttttgtcatgcatagaggataaaaatccttgtatattttttgaacctaaaatactttacagggcagcagcggaagaagtccctatagaaccatacaacatcccactgtcccaggccgaagtcatacaggaagggagtgatgttactctagttgcctggggcactcaggttcatgtgatccgagaggtagcttccatggcaaaagaaaagcttggagtgtcttgtgaagtcattgatctgaggactataataccttgggatgtggacacaatttgtaagtctgtgatcaaaacagggcgactgctaatcagtcacgaggctcccttgacaggcggctttgcatcggaaatcagctctacagttcaggaggaatgtttcttgaacctagaggctcctatatcaagagtatgtggttatgacacaccatttcctcacatttttgaaccattctacatcccagacaaatggaagtgttatgatgcccttcgaaaaatgatcaactattga

SEQ ID NO: 3 - protein, 445 aa, Homo sapiens
  SIGNAL 1..45
MAVAIAAARVWRLNRGLSQAALLLLRQPGARGLARSHPPRQQQQFSSLDDKPQFPGASAEFIDKLEFIQPNVISGIPIYRVMDRQGQIINPSEDPHLPKEKVLKLYKSMTLLNTMDRILYESQRQGRISFYMTNYGEEGTHVGSAAALDNTDLVFGQYREAGVLMYRDYPLELFMAQCYGNISDLGKGRQMPVHYGCKERHFVTISSPLATQIPQAVGAAYAAKRANANRVVICYFGEGAASEGDAHAGFNFAATLECPIIFFCRNNGYAISTPTSEQYRGDGIAARGPGYGIMSIRVDGNDVFAVYNATKEARRRAVAENQPFLIEAMTYRIGHHSTSDDSSAYRSVDEVNYWDKQDHPISRLRHYLLSQGWWDEEQEKAWRKQSRRKVMEAFEQAERKPKPNPNLLFSDVYQEMPAQLRKQQESLARHLQTYGEHYPLDHFDK

SEQ ID NO: 4 - DNA, 1338 bp, Homo sapiens
  source 1..1338: /mol_type=“unassigned DNA” /organism=“Homo sapiens”
atggcggtagcgatcgctgcagcgagggtctggcggctaaaccgtggtttgagccaggctgccctcctgctgctgcggcagcctggggctcggggactggctagatctcacccccccaggcagcagcagcagttttcatctctggatgacaagccccagttcccaggggcctcggcggagtttatagataagttggaattcatccagcccaacgtcatctctggaatccccatctaccgcgtcatggaccggcaaggccagatcatcaaccccagcgaggacccccacctgccgaaggagaaggtgctgaagctctacaagagcatgacactgcttaacaccatggaccgcatcctctatgagtctcagcggcagggccggatctccttctacatgaccaactatggtgaggagggcacgcacgtggggagtgccgccgccctggacaacacggacctggtgtttggccagtaccgggaggcaggtgtgctgatgtatcgggactaccccctggaactattcatggcccagtgctatggcaacatcagtgacttgggcaaggggcgccagatgcctgtccactacggctgcaaggaacgccacttcgtcactatctcctctccactggccacgcagatccctcaggcggtgggggcggcgtacgcagccaagcgggccaatgccaacagggtcgtcatctgttacttcggcgagggggcagccagtgagggggacgcccatgccggcttcaacttcgctgccacacttgagtgccccatcatcttcttctgccggaacaatggctacgccatctccacgcccacctctgagcagtatcgcggcgatggcattgcagcacgaggccccgggtatggcatcatgtcaatccgcgtggatggtaatgatgtgtttgccgtatacaacgccacaaaggaggcccgacggcgggctgtggcagagaaccagcccttcctcatcgaggccatgacctacaggatcgggcaccacagcaccagtgacgacagttcagcgtaccgctcggtggatgaggtcaattactgggataaacaggaccaccccatctcccggctgcggcactatctgctgagccaaggctggtgggatgaggagcaggagaaggcctggaggaagcagtcccgcaggaaggtgatggaggcctttgagcaggccgagcggaagcccaaacccaaccccaacctactcttctcagacgtgtatcaggagatgcccgcccagctccgcaagcagcaggagtctctggcccgccacctgcagacctacggggagcactacccactggatcacttcgataagtga

SEQ ID NO: 5 - protein, 411 aa, Homo sapiens
  SIGNAL 1..30
MILASVLRSGPGGGLPLRPLLGPALALRARSTSATDTHHVEMARERSKTVTSFYNQSAIDAAAEKPSVRLTPTMMLYAGRSQDGSHLLKSARYLQQELPVRIAHRIKGFRCLPFIIGCNPTILHVHELYIRAFQKLTDFPPIKDQADEAQYCQLVRQLLDDHKDVVTLLAEGLRESRKHIEDEKLVRYFLDKTLTSRLGIRMLATHHLALHEDKPDFVGIICTRLSPKKIIEKWVDFARRLCEHKYGNAPRVRINGHVAARFPFIPMPLDYILPELLKNAMRATMESHLDTPYNVPDVVITIANNDVDLIIRISDRGGGIAHKDLDRVMDYHFTTAEASTQDPRISPLFGHLDMHSGAQSGPMHGFGFGLPTSRAYAEYLGGSLQLQSLQGIGTDVYLRLRHIDGREESFR

SEQ ID NO: 6 - DNA, 1239 bp, Homo sapiens
  source 1..1239: /mol_type=“unassigned DNA” /organism=“Homo sapiens”
atgatcctggcgtcggtgctgaggagcggtcccgggggcgggcttccgctccggcccctcctgggacccgcactcgcgctccgggcccgctcgacgtcggccaccgacacacaccacgtggagatggctcgggagcgctccaagaccgtcacctccttttacaaccagtcggccatcgacgcggcagcggagaagccctcagtccgcctaacgcccaccatgatgctctacgctggccgctctcaggacggcagccaccttctgaaaagtgctcggtacctgcagcaagaacttccagtgaggattgctcaccgcatcaagggcttccgctgccttcctttcatcattggctgcaaccccaccatactgcacgtgcatgagctatatatccgtgccttccagaagctgacagacttccctccgatcaaggaccaggcggacgaggcccagtactgccagctggtgcgacagctgctggatgaccacaaggatgtggtgaccctcttggcagagggcctacgtgagagccggaagcacatagaggatgaaaagctcgtccgctacttcttggacaagacgctgacttcgaggcttggaatccgcatgttggccacgcatcacctggcgctgcatgaggacaagcctgactttgtcggcatcatctgtactcgtctctcaccaaagaagattattgagaagtgggtggactttgccagacgcctgtgtgagcacaagtatggcaatgcgccccgtgtccgcatcaatggccatgtggctgcccggttccccttcatccctatgccactggactacatcctgccggagctgctcaagaatgccatgagagccacaatggagagtcacctagacactccctacaatgtcccagatgtggtcatcaccatcgccaacaatgatgtcgatctgatcatcaggatctcagaccgtggtggaggaatcgctcacaaagatctggaccgggtcatggactaccacttcactactgctgaggccagcacacaggacccccggatcagccccctctttggccatctggacatgcatagtggcgcccagtcaggacccatgcacggctttggcttcgggttgcccacgtcacgggcctacgcggagtacctcggtgggtctctgcagctgcagtccctgcagggcattggcacggacgtctacctgcggctccgccacatcgatggccgggaggaaagcttccggatctga